(19) United States
(12) Patent Application Publication
(10) Pub. No.: US 2024/0057591 A1
(43) Pub. Date: Feb. 22, 2024

(54) ANTIMICROBIAL WET WIPES

(71) Applicant: Kimberly-Clark Worldwide, Inc., Neenah, WI (US)

(72) Inventors: David W. Koenig, Menasha, WI (US); Salvador E. Escobar, Appleton, WI (US); Andrew R. Kischnick, Appleton, WI (US); Brian T. Roessler, Cumming, GA (US); Jane Ballesteros, Oshkosh, WI (US)

(21) Appl. No.: 18/547,596

(22) PCT Filed: Mar. 30, 2022
(86) PCT No.: PCT/US2022/022496

§ 371 Date: Aug. 23, 2023

Related U.S. Application Data
(60) Provisional application No. 63/168,589, filed on Mar. 31, 2021.

(51) Int. Cl. A01N 25/34; A01P 1/00; A01N 59/04

(52) U.S. Cl. CPC A01N 25/34; A01P 1/00; A01N 59/04

(57) ABSTRACT

Described herein is an antimicrobial wet wipe including a wet wipe formulation. Also described herein is a method of preserving the wet wipe formulation. Also described herein is a method of manufacturing the wet wipe formulation.

### CROSS-REFERENCE TO RELATED APPLICATIONS

This application claims priority to U.S. Provisional Application Ser. No. 63/168,589, filed on Mar. 31, 2021, the content of which is hereby incorporated by reference in its entirety.

### FIELD OF DISCLOSURE

This disclosure is directed to antimicrobial wet wipe compositions, methods of preserving wet wipe formulations, and methods of manufacturing thereof.

### BACKGROUND

Maintaining microbial water quality for the manufacture of wet wipes is critical for release of wet wipe products to consumers. Currently, ozonation, ultraviolet (UV) radiation, chlorination, and filtration are used to reduce bioburden and/or preserve process water used in manufacturing of wet wipe formulations. Most process water systems in a manufacturing environment can involve long runs of piping, large holding tanks, multiple pumps, and dead legs that work against maintaining adequate levels of appropriate disinfectant and increase the potential for contamination. Increased bioburden in the water can lead to rejection of substantial quantities of finished product due to safety concerns for the consumer. Therefore, there is a need for simple, robust, and effective means of preserving water or other aqueous solutions used to make wet wipes.

Additionally, preservative-free wet wipe formulations are preferred by consumers. Adding chemicals to preserve products such as wet wipes have a negative consumer perception as well as possible regulatory hurdles. Therefore, there is a need for a safe and effective preservative for a wet wipe formulation.

In general, it has been reported that carbon dioxide (CO₂) can be used to preserve food; however, it is unknown whether carbon dioxide or other gases are capable of preserving wet solutions during manufacturing.

JP 2016165431 demonstrates impregnation of nanobubbles of gases in wiping disinfection sheets at atmospheric pressures. However, it does not address solutions including solubilized gases at increased pressures for the preservation of wet wipes.

It was surprisingly found in the present disclosure that a pressurized gas comprising an inert gas, such as carbon dioxide, may be used to reduce or eliminate microbial contamination in the water treatment and solution mix systems used to make wet wipe formulations.

Adding a pressurized gas comprising an inert gas, such as carbon dioxide, into a water system at the source, before and after reverse osmosis and/or ultrafiltration, may reduce and eliminate microbes in process water. The pressurized gas composition comprising an inert gas may eliminate the need to add chlorine and ozone to process water, as well as the steps required to maintain levels of these chemical preservatives and eliminate them as appropriate during the process. The pressurized gas composition comprising an inert gas may also be added to a wet wipe formulation to reduce and eliminate microbial contamination in the wet wipe formulation and the final wet wipe product, thereby providing a preservative-free formulation, increasing foaming, and providing a visual cue of bubbles as a consumer product attribute.

Described herein are antimicrobial wet wipe compositions, methods of preserving wet wipe formulations, and methods of manufacturing thereof. The compositions and methods may be used to reduce or eliminate microbial contamination in wet wipe formulations and compositions comprising wet wipe formulations.

### Objective of the Disclosure

The aim of the present disclosure is to provide compositions and methods for reducing or eliminating microbial contamination in wet wipe formulations.

### Brief Description of the Disclosure

In one aspect, provided herein is an antimicrobial wet wipe composition, the composition comprising a wet wipe formulation, the wet wipe formulation comprising an aqueous solution, a pressurized gas composition comprising an inert gas solubilized in the aqueous solution, and optionally, at least one additive.

In another aspect, provided herein is a method of preserving a wet wipe formulation, the method comprising solubilizing a pressurized gas composition comprising an inert gas in the wet wipe formulation.

In yet another aspect, provided herein is a method of manufacturing a wet wipe formulation, the method comprising solubilizing a pressurized gas composition comprising an inert gas in an aqueous solution and adding the aqueous solution to the wet wipe formulation.

### BRIEF DESCRIPTION OF THE DRAWINGS

FIG. 1 is an exemplary embodiment depicting a perspective view of a wet wipe substrate in accordance with the present disclosure.

FIG. 2 is an exemplary embodiment depicting a diagram of injection points for CO₂ into a water treatment system used for manufacturing of wet wipes in accordance with the present disclosure. CO₂ gas may be injected into the ‘softened water’ stream before the filtration stage. Given that the system is pressurized, CO₂ losses are minimal before the points of use.

FIG. 3 is an exemplary embodiment depicting a diagram of injection points for CO₂ into a solution mix and buffer tanks used for the manufacturing of wet wipes in accordance with the present disclosure. The CO₂ gas can be injected into the tank using a micro-bubble diffuser to maximize gas absorption.

FIG. 4A is an exemplary embodiment depicting reduction in bacterial load in contaminated reverse osmosis water upon adding 300 psi of CO₂ in accordance with the present disclosure.

FIG. 4B is an exemplary embodiment depicting the log average reduction in bacterial load in contaminated reverse osmosis water upon adding 300 psi of CO₂ in accordance with the present disclosure.

FIG. 5 is an exemplary embodiment depicting a graph of pH measurements of 100 mL of preservative free wet wipe formulation after addition of 8.2 g of CO₂, and subsequent exposure to 1 atm (20° C.) in accordance with the present disclosure.

### DETAILED DESCRIPTION OF THE DISCLOSURE

Generally, antimicrobial wet wipe compositions according to the present disclosure comprise a wet wipe formulation. In many embodiments, the antimicrobial wet wipe composition comprises a wet wipe substrate. In many embodiments, the wet wipe formulation comprises an aqueous solution, a pressurized gas composition that is solubilized in the aqueous solution, and optionally, at least one additive. In many embodiments, the pressurized gas composition comprises an inert gas.

As used herein, antimicrobial generally refers to the property of killing microorganisms or inhibiting their growth. Antimicrobial properties may include antibacterial, antifungal, and/or antiviral properties. Microorganisms may include bacteria, fungi, and/or viruses. Non-limiting examples of microorganisms include any microorganisms belonging to the genera Burkholderia, Staphylococcus, Escherichia, Pseudomonas, or Candida. In some embodiments, the microorganisms are Burkholderia cepacia, Staphylococcus aureus, Escherichia coli, Pseudomonas aeruginosa, or Candida albicans.

Pressurized Gas

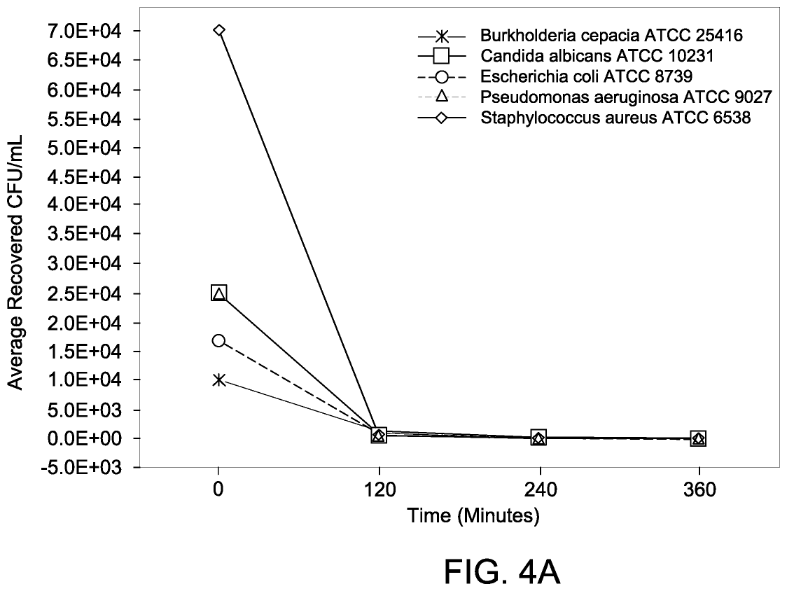

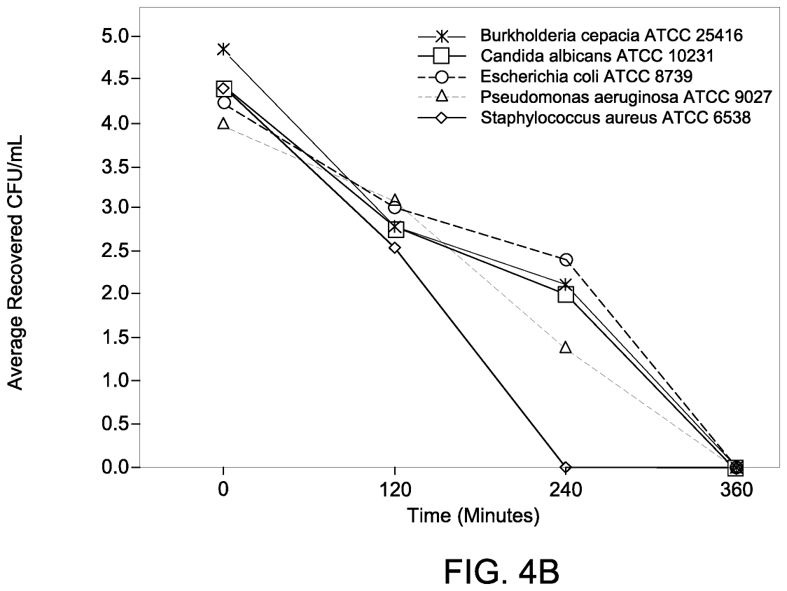

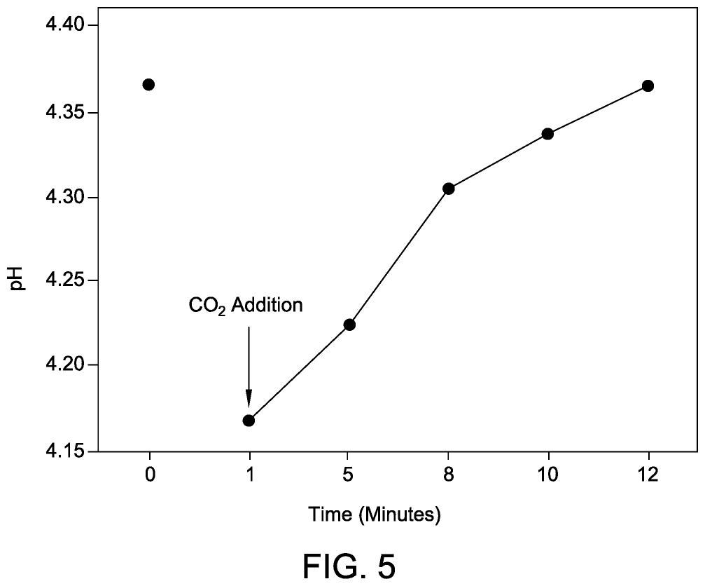

Solubilizing the pressurized gas composition comprising an inert gas reduces or eliminates microbial growth in the aqueous solution, the wet wipe formulation, and/or the antimicrobial wet wipe composition. For example, as shown in FIG. 4A and FIG. 4B, solubilizing a pressurized gas composition comprising carbon dioxide greatly reduces microbial growth in contaminated water. Without being bound by theory, reduction or elimination of microbial growth may be effected by the increase in pressure and/or reduction in pH that occurs upon solubilizing the pressurized gas composition. For example, FIG. 5 shows the reduction in pH of a preservative-free wet wipe formulation after solubilizing a pressurized gas composition comprising carbon dioxide.

The inert gas comprised in the pressurized gas composition may be any suitable gas known in the art that is not substantially chemically reactive. Non-limiting examples include noble gases, such as helium (He), neon (Ne), argon (Ar), krypton (Kr), xenon (Xe), and radon (Rn), and inert molecular gases, such as nitrogen (N₂), and carbon dioxide (CO₂). In some embodiments, the inert gas is selected from the group consisting of carbon dioxide, nitrogen, argon, helium, and mixtures thereof. In some preferred embodiments, the inert gas is carbon dioxide. In some preferred embodiments, the inert gas is a mixture of carbon dioxide and nitrogen.

The inert gas may be present in the aqueous solution and/or wet wipe formulation at a range of suitable concentrations. Concentrations are defined herein in moles per liter (mol/L). In many embodiments, the aqueous solution and/or wet wipe formulation comprises the inert gas at a concentration in a range of from about 0.01 mol/L, about 0.05 mol/L, about 0.10 mol/L, about 0.15 mol/L, about 0.20 mol/L, about 0.25 mol/L, about 0.30 mol/L, about 0.35 mol/L, about 0.40 mol/L, about 0.45 mol/L, about 0.50 mol/L, about 0.55 mol/L, about 0.60 mol/L, about 0.65 mol/L, or about 0.70 mol/L, to about 0.01 mol/L, about 0.05 mol/L, about 0.10 mol/L, about 0.15 mol/L, about 0.20 mol/L, about 0.25 mol/L, about 0.30 mol/L, about 0.35 mol/L, about 0.40 mol/L, about 0.45 mol/L, about 0.50 mol/L, about 0.55 mol/L, about 0.60 mol/L, about 0.65 mol/L, or about 0.70 mol/L.

In some embodiments, the aqueous solution and/or wet wipe formulation comprises the inert gas at a concentration in a range of from about 0.01 mol/L to about 0.30 mol/L. In some embodiments, the aqueous solution and/or wet wipe formulation comprises the inert gas at a concentration in a range of from about 0.01 mol/L to about 0.50 mol/L. In some embodiments, the aqueous solution and/or wet wipe formulation comprises the inert gas at a concentration in a range of from about 0.01 mol/L to about 0.70 mol/L. In some embodiments, the aqueous solution and/or wet wipe formulation comprises the inert gas at a concentration in a range of from about 0.30 mol/L to about 0.50 mol/L. In some embodiments, the aqueous solution and/or wet wipe formulation comprises the inert gas at a concentration in a range of from about 0.30 mol/L to about 0.70 mol/L. In some embodiments, the aqueous solution and/or wet wipe formulation comprises the inert gas at a concentration in a range of from about 0.50 mol/L to about 0.70 mol/L.

The pressurized gas composition comprising an inert gas may be present in the aqueous solution and/or the wet wipe formulation at a range of suitable pressures. Pressures are defined herein in pound-force per square inch (psi). In many embodiments, the aqueous solution and/or wet wipe formulation comprises the pressurized gas composition at a pressure in a range of from about 15 psi, about 20 psi, about 30 psi, about 40 psi, about 50 psi, about 60 psi, about 70 psi, about 80 psi, about 90 psi, about 100 psi, about 110 psi, about 120 psi, about 130 psi, about 140 psi, about 150 psi, about 160 psi, about 170 psi, about 180 psi, about 190 psi, about 200 psi, about 210 psi, about 220 psi, about 230 psi, about 240 psi, about 250 psi, about 260 psi, about 270 psi, about 280 psi, about 290 psi, or about 300 psi to about 15 psi, about 20 psi, about 30 psi, about 40 psi, about 50 psi, about 60 psi, about 70 psi, about 80 psi, about 90 psi, about 100 psi, about 110 psi, about 120 psi, about 130 psi, about 140 psi, about 150 psi, about 160 psi, about 170 psi, about 180 psi, about 190 psi, about 200 psi, about 210 psi, about 220 psi, about 230 psi, about 240 psi, about 250 psi, about 260 psi, about 270 psi, about 280 psi, about 290 psi, or about 300 psi.

In some embodiments, the aqueous solution and/or wet wipe formulation comprises the pressurized gas composition at a pressure in a range of from about 15 psi to about 300 psi. In some embodiments, the aqueous solution and/or wet wipe formulation comprises the pressurized gas at a pressure in a range of from about 15 psi to about 100 psi. In some embodiments, the aqueous solution and/or wet wipe formulation comprises the pressurized gas at a pressure in a range of from about 15 psi to about 200 psi. In some embodiments, the aqueous solution and/or wet wipe formulation comprises the pressurized gas at a pressure in a range of from about 100 psi to about 300 psi.

As discussed herein, the pressurized gas composition comprising an inert gas may modulate the pH of the aqueous solution and/or wet wipe formulation and contribute to antimicrobial properties. The inert gas may either increase pH or decrease pH. In many preferred embodiments, the inert gas decreases the pH of the aqueous solution and/or wet wipe formulation.

For example, as shown in FIG. 5, solubilizing a pressurized gas composition comprising carbon dioxide in a wet wipe formulation reduces pH by approximately 0.20 pH units. In many embodiments, solubilizing the pressurized gas composition comprising an inert gas modulates the pH of the aqueous solution and/or wet wipe formulation by about 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 pH units.

The aqueous solution and/or wet wipe formulation may have any pH value within a suitable range. In many embodiments, the aqueous solution and/or wet wipe formulation has a pH in a range from about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, or about 14.0 to about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, or about 14.0.

In some embodiments, the aqueous solution and/or wet wipe formulation has a pH in a range from about 3.0, about 3.5, about 4.0, about 4.5, about 5.0, about 5.5, about 6.0, about 6.5, about 7.0, about 7.5, or about 8.0, to about 3.5, about 4.0, about 4.5, about 5.0, about 5.5, about 6.0, about 6.5, about 7.0, about 7.5, or about 8.0.

It may be desirable to modulate the temperature of the aqueous solution and/or wet wipe formulation, as it is known that temperature affects the solubility of any given gas in solution. The aqueous solution and/or wet wipe formulation may be comprised at any range of suitable temperatures. Temperatures are defined herein in degrees Celsius (° C.). The aqueous solution or wet wipe formulation may be comprised at a temperature in a range of from about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C., to about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C.

The pressurized gas composition comprising an inert gas may be solubilized in any aqueous solution suitable for use in a wet wipe formulation. As used herein, an aqueous solution is a solution that comprises water as the primary component. In some preferred embodiments, the aqueous solution consists essentially of water. As used herein, an aqueous solution consisting essentially of water includes water and optionally small amounts of impurities typically found in water. These impurities may be, for example, salts, minerals, metals, dissolved and/or suspended solids, and combinations thereof. These impurities may also include any water treatment chemicals typically found in municipal water supplies, such as chlorine and chlorine compounds, fluoride, ozone, organic polymers, sodium hydroxide, and combinations thereof.

When the pressurized gas composition comprising an inert gas is solubilized in an aqueous solution, it may alter the conductivity of the aqueous solution. For example, it may increase the conductivity of the aqueous solution, consequently altering the purity of the aqueous solution. In some embodiments, the aqueous solution consists essentially of water having a purity selected from the group consisting of purified water, highly purified water, water for injection, deionized water, reverse osmosis water, and combinations thereof. In some preferred embodiments, the aqueous solution consists essentially of water having a purity of reverse osmosis water.

In some embodiments, it may be desirable to remove the pressurized gas composition comprising an inert gas from the aqueous solution and/or wet wipe formulation. For example, removal of the pressurized gas composition comprising an inert gas may be desired prior to packaging the wet wipe composition comprising the wet wipe formulation, or prior to formulating a consumer product comprising the wet wipe composition. This can be easily achieved by exposing the aqueous solution and/or wet wipe formulation to atmospheric conditions at room temperature, for example. FIG. 5 shows that after exposure to atmospheric conditions, a pressurized gas composition comprising carbon dioxide solubilized in a wet wipe formulation is quickly dissipated from the wet wipe formulation.

Wet Wipe Substrate

In many embodiments, the antimicrobial wet wipe composition comprises a wet wipe substrate. Generally, the wet wipe substrate may be any suitable wet wipe substrate known in the art. For example, the wet wipe substrate may include or be in the form of woven materials, non-woven materials, and/or fibrous materials. As used herein, a wet wipe substrate is any suitable wettable substrate that can serve as a delivery vehicle for the wet wipe formulation. For example, the wet wipe formulation may be incorporated into or onto a wettable substrate, such as a wipe substrate, an absorbent substrate, a fabric or cloth substrate, a tissue or paper towel substrate, or the like. In some embodiments, the wet wipe formulation may be used in combination with a wipe substrate to form a wet wipe or may be a wetting composition for use in combination with a wipe which may be dispersible. In other embodiments, the wet wipe formulation may be incorporated into wipes of wipe-based consumer products such as wet wipes, hand wipes, face wipes, cosmetic wipes, cloths and the like.

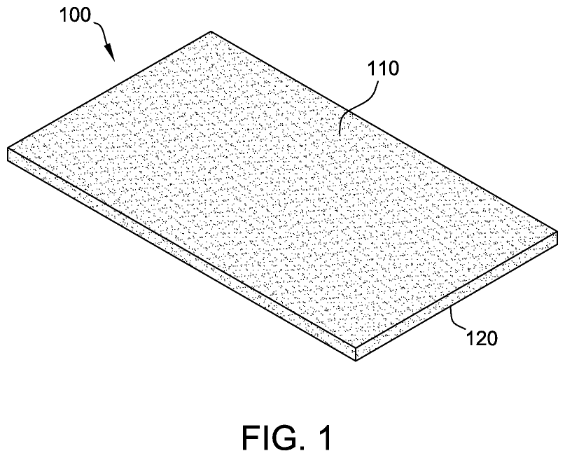

FIG. 1 shows an exemplary embodiment of a wet wipe substrate indicated generally at 100 having a top or front surface 110, and a bottom or back surface 120 in accordance with the present disclosure. In one embodiment, the wet wipe substrate 100 is wetted with the wet wipe formulation described herein.

The antimicrobial wet wipe compositions according to the present disclosure are particularly useful in consumer products and/or personal care products. The antimicrobial wet wipe compositions may be incorporated into any suitable consumer product known in the art. Examples of personal care products include wet wipes, hand wipes, face wipes, baby wipes, cosmetic wipes, feminine hygiene wipes, cooling wipes, cleaning wipes, flushable wipes, and combinations thereof.

In some embodiments, the antimicrobial wet wipe compositions according to the present disclosure are incorporated into personal care products including flushable wet wipes. Flushable wet wipes as used herein are wet wipes designed to be flushed down a toilet or otherwise disposed of into a sewer system or septic system without clogging or damaging said sewer system or septic system. Flushable wet wipes may be comprised of fibers that are completely biodegradable, such that the fibers break down or disintegrate after flushing or otherwise being disposed of.

The wet wipe substrate may comprise a nonwoven material that is capable of being wetted with the wet wipe formulation disclosed herein. As used herein, the nonwoven material comprises a fibrous material, where the fibrous material comprises a sheet that has a structure of individual fibers or filaments randomly arranged in a mat-like fashion. Nonwoven materials may be made from a variety of processes including, but not limited to, airlaid processes, wet-laid processes such as with cellulosic-based tissues or towels, hydroentangling processes, staple fiber carding and bonding, melt blown, and solution spinning.

The fibers forming the fibrous material may be made from a variety of materials including natural fibers, synthetic fibers, and combinations thereof. The choice of fibers may depend upon, for example, the intended end use of the finished wet wipe composition and the fiber cost. For instance, suitable fibers may include, but are not limited to, natural fibers such as cotton, linen, jute, hemp, wool, wood pulp, etc. Similarly, suitable fibers may also include regenerated cellulosic fibers, such as viscose rayon and cuprammonium rayon; modified cellulosic fibers, such as cellulose acetate; or synthetic fibers, such as those derived from polypropylenes, polyethylenes, polyolefins, polyesters, polyamides, polyacrylics, etc. Regenerated cellulose fibers include rayon in all its varieties as well as other fibers derived from viscose or chemically modified cellulose, including regenerated cellulose and solvent-spun cellulose, such as Lyocell. Among wood pulp fibers, any known papermaking fibers may be used, including softwood and hardwood fibers. Fibers, for example, may be chemically pulped or mechanically pulped, bleached or unbleached, virgin or recycled, high yield or low yield, and the like. Chemically treated natural cellulosic fibers may be used, such as mercerized pulps, chemically stiffened or crosslinked fibers, or sulfonated fibers.

In addition, cellulose produced by microbes and other cellulosic derivatives may be used. As used herein, the term “cellulosic” is meant to include any material having cellulose as a major constituent, and, specifically, comprising at least 50 percent by weight cellulose or a cellulose derivative. Thus, the term includes cotton, typical wood pulps, non-woody cellulosic fibers, cellulose acetate, cellulose triacetate, rayon, thermomechanical wood pulp, chemical wood pulp, debonded chemical wood pulp, milkweed, or bacterial cellulose. Blends of one or more of any of the previously described fibers may also be used, if so desired.

The fibrous material may be formed from a single layer or multiple layers. In the case of multiple layers, the layers are generally positioned in a juxtaposed or surface-to-surface relationship and all or a portion of the layers may be bound to adjacent layers. The fibrous material may also be formed from a plurality of separate fibrous materials wherein each of the separate fibrous materials may be formed from a different type of fiber.

Airlaid nonwoven fabrics are particularly well suited for use as wet wipes. The basis weights for airlaid nonwoven fabrics may range from about 20 to about 200 grams per square meter (gsm) with staple fibers having a denier of about 0.5 to about 10 and a length of about 6 to about 15 millimeters. Wet wipes may generally have a fiber density of about 0.025 g/cc to about 0.2 g/cc. Wet wipes may generally have a basis weight of about 20 gsm to about 150 gsm. More desirably the basis weight may be from about 30 to about 90 gsm. Even more desirably the basis weight may be from about 50 gsm to about 75 gsm.

Processes for producing airlaid non-woven basesheets are described in, for example, published U.S. Pat. App. Pub. No. 2006/0008621, herein incorporated by reference to the extent it is consistent herewith.

Preservatives

The pressurized gas composition comprising an inert gas allows for the aqueous solution or wet wipe formulation according to the present disclosure to be substantially free of any preservative, yet still provides adequate efficacy against bacterial growth. Thus, in some embodiments, the aqueous solution and/or wet wipe formulation does not include a preservative. One advantage of a preservative-free wet wipe formulation is that the wet wipe formulation is microbiome and skin friendly. Eliminating or reducing the use of preservatives may also provide environmental benefits, for example, by reducing the need for harsh chemicals. Further, eliminating or reducing the use of preservatives may also reduce the cost of the wet wipe formulation and make it easier to develop stable formulations without the use of significant concentrations of surfactants or emulsifiers.

However, in some embodiments, the aqueous solution and/or wet wipe formulation may include any of the various preservatives discussed herein in addition to the pressurized gas composition comprising an inert gas. In some embodiments, the pressurized gas comprising an inert gas allows for a reduction in the effective amount of preservative needed to prevent microbial growth in the aqueous solution and/or wet wipe formulation, compared to an aqueous solution and/or wet wipe formulation that does not include the pressurized gas comprising an inert gas.

A preservative in accordance with the present disclosure may be, for example, a chemical preservative. As used herein, a chemical preservative is a compound that has been historically used for treatment of water and/or aqueous solutions in a manufacturing process. Non-limiting examples of chemical preservatives include ozone, chlorine, bromine, iodine, silver, other halogens, chlorine dioxide, n-halamines, and mixtures thereof. Preservatives, such as the chemical preservatives described herein, are often consumed during the manufacturing process. An advantage of using the pressurized gas composition comprising an inert gas in accordance with the present disclosure is that the levels of the gas are not consumed and thus remain constant during the manufacturing process.

A preservative may also be a traditional preservative. As used herein, a traditional preservative is any compound that has been historically recognized by regulatory bodies as providing preservative or antimicrobial effect, such as those listed in the European Union's Annex V list of preservatives allowed in cosmetics products. Non-limiting examples of traditional preservatives include, but are not limited to: propionic acid and salts thereof, salicylic acid and salts thereof; sorbic acid and salts thereof, benzoic acid and salts and esters thereof, formaldehyde; paraformaldehyde; o-phenylphenol and salts thereof, zinc pyrithione; inorganic sulfites; hydrogen sulfites; chlorobutanol; benzoic parabens, such as methylparaben, propylparaben, butylparaben, ethylparaben, isopropylparaben, isobutylparaben, benzylparaben, sodium methylparaben and sodium propylparaben; dehydroacetic acid and salts thereof, formic acid and salts thereof, dibromohexamidine isethionate; thimerosal; phenylmercuric salts; undecylenic acid and salts thereof, hexetidine; 5-bromo-5-nitro-1,3-dioxane; 2-bromo-2-nitropropane-1,3,-diol; dichlorobenzyl alcohol; triclocarban; p-chloro-m-cresol; triclosan; chloroxylenol; imidazolidinyl urea; polyaminopropyl biguanide; phenoxyethanol, methenamine; quaternium-15; climbazole; DMDM hydantoin; benzyl alcohol; piroctone olamine; bromochlorophene; o-cymen-5-ol; methylchloroisothiazolinone; methylisothiazolinone; chlorophene; chloroacetamide; chlorhexidine; chlorhexidine diacetate; chlorhexidine digluconate; chlorhexidine dihydrochloride; phenoxyisopropanol; alkyl (C12-C22) trimethyl ammonium bromide and chlorides; dimethyl oxazolidine; diazolidinyl urea; hexamidine; hexamidine diisethionate; glutaral; 7-ethylbicyclooxazolidine; chlorphenesin; sodium hydroxymethylglycinate; silver chloride; benzethonium chloride; benzalkonium chloride; benzalkonium bromide; benzylhemiformal; iodopropynyl butylcarbamate; ethyl lauroyl arginate HCl; citric acid; and silver citrate. The chemical preservatives described above are included within the category of traditional preservatives.

A preservative may also be a non-traditional preservative. As used herein, a non-traditional preservative is any compound that is known to exhibit antimicrobial effects in addition to its primary function, but that has not historically been recognized as a preservative by regulatory bodies (such as on the European Union's Annex V list). Non-limiting examples of non-traditional preservatives include, but are not limited to, hydroxyacetophenone, caprylyl glycol, sodium coco-PG dimonium chloride phosphate, phenylpropanol, lactic acid and salts thereof, caprylhydroxamic acid, levulinic acid and salts thereof, sodium lauroyl lactylate, phenethyl alcohol, sorbitan caprylate, glyceryl caprate, glyceryl caprylate, ethylhexylglycerin, p-anisic acid and salts thereof, gluconolactone, decylene glycol, 1,2-hexanediol, glucose oxidase and lactoperoxidase, leuconostoc/radish root ferment filtrate and glyceryl laurate.

In some embodiments, the antimicrobial wet wipe composition also comprises a coform or other material that includes a bound antimicrobial. Non-limiting examples include polyhexamethylene biguanide, silver, copper, and silane (SiH4) quaternary ammonia compounds.

In some embodiments, the coform or bound antimicrobial may be used in conjunction with a wet wipe formulation that does not include a preservative. Typically, wet wipe formulations have required the inclusion of a preservative from the supplier to ensure microbial quality of the raw material during transportation to the mill and storage. However, the wet wipe formulation comprising a pressurized gas composition described herein allows for exclusion of preservatives from the antimicrobial wet wipe, if desired. In some embodiments, the coform or bound antimicrobial attached to the wet wipe substrate may provide the necessary preservation of the wet wipe during consumer use.

Additives

The wet wipe formulation described herein may include any additive conventionally found in cosmetic, pharmaceutical, medical, or personal care compositions/products in an established fashion and at established levels. For example, the wet wipe formulation may comprise additional compatible pharmaceutically active and compatible materials for combination therapy, such as antioxidants, anti-parasitic agents, antipruritics, antifungals, antiseptic actives, biological actives, astringents, keratolytic actives, local anaesthetics, anti-stinging agents, anti-reddening agents, skin soothing agents, external analgesics, film formers, skin exfoliating agents, sunscreens, and combinations thereof.

Other suitable additives that may be included in the wet wipe formulation of the present disclosure include compatible colorants, deodorants, emulsifiers, anti-foaming agents (when foam is not desired), lubricants, skin conditioning agents, skin protectants and skin benefit agents (e.g., aloe vera and tocopheryl acetate), solvents, solubilizing agents, suspending agents, wetting agents, pH adjusting ingredients, chelators, propellants, dyes and/or pigments, and combinations thereof.

Another component that may be suitable for addition to the wet wipe formulation is a fragrance. Any compatible fragrance may be used. Typically, the fragrance is present in an amount from about 0% (by weight of the composition) to about 5% (by weight of the composition), and more typically from about 0.01% (by weight of the composition) to about 3% (by weight of the composition). In one desirable embodiment, the fragrance will have a clean, fresh and/or neutral scent to create an appealing delivery vehicle for the end consumer.

Organic sunscreens that may be present in the wet wipe formulation include ethylhexyl methoxycinnamate, avobenzone, octocrylene, benzophenone-4, phenylbenzimidazole sulfonic acid, homosalate, oxybenzone, benzophenone-3, ethylhexyl salicylate, and mixtures thereof.

Methods

The present disclosure also provides for a method of preserving a wet wipe formulation. Generally, the method of preserving a wet wipe formulation in accordance with the present disclosure comprises solubilizing a pressurized gas composition comprising an inert gas in the wet wipe formulation. The pressurized gas composition comprising an inert gas and the wet wipe formulation are as discussed in accordance with the present disclosure.

As used herein, “preserving” generally refers to a means of killing microorganisms or inhibiting their growth. In other words, the methods of preserving a wet wipe formulation in accordance with the present disclosure confer antimicrobial properties to the wet wipe formulation.

The present disclosure also provides for a method of manufacturing a wet wipe formulation. Generally, the method of manufacturing a wet wipe formulation in accordance with the present disclosure comprises solubilizing a pressurized gas composition comprising an inert gas in an aqueous solution, and adding the aqueous solution to the wet wipe formulation. The wet wipe formulation, pressurized gas composition comprising an inert gas, and the aqueous solution are as discussed in accordance with the present disclosure.

In some embodiments, conventional methods of purification or treatment are applied to the aqueous solution before or after solubilizing the pressurized gas composition comprising an inert gas. For example, filtration, reverse osmosis, or UV treatment may be applied to the aqueous solution. One advantage of the methods of manufacturing a wet wipe formulation provided herein is that they are compatible with conventional methods of purification or treatment.

In some embodiments, the pressurized gas composition comprising an inert gas is used to preserve or reduce or eliminate microbial contamination in the water treatment and/or solution mix systems used to manufacture wet wipe formulations. The pressurized gas composition comprising an inert gas may be added to the water treatment system before, concurrently, or after applying conventional water treatments.

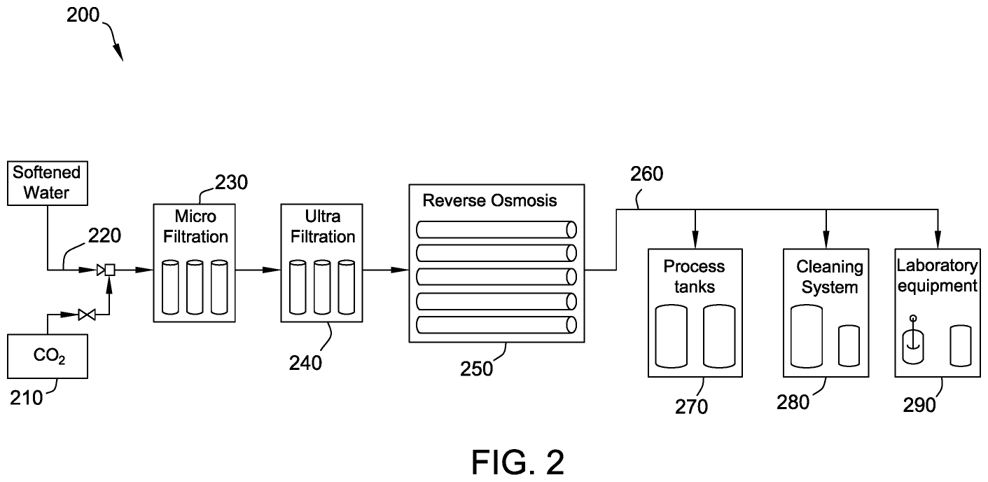

For example, the pressurized gas composition comprising an inert gas may be added before, concurrently, or after a reverse osmosis, filtration, or ultraviolet (UV) treatment step. FIG. 2 shows an exemplary schematic of a water treatment system 200 for manufacturing a wet wipe formulation. A pressurized gas composition comprising carbon dioxide 210 is injected into a softened water stream 220 prior to filtration. The softened water stream 220 including the pressurized gas composition comprising carbon dioxide 210 then undergoes a microfiltration step 230, ultrafiltration step 240, and reverse osmosis step 250 to yield purified water 260. In some embodiments, the purified water 260 is transferred to various systems used in the methods of manufacturing in accordance with the present disclosure, such as process tanks 270, cleaning system 280, and/or laboratory equipment 290.

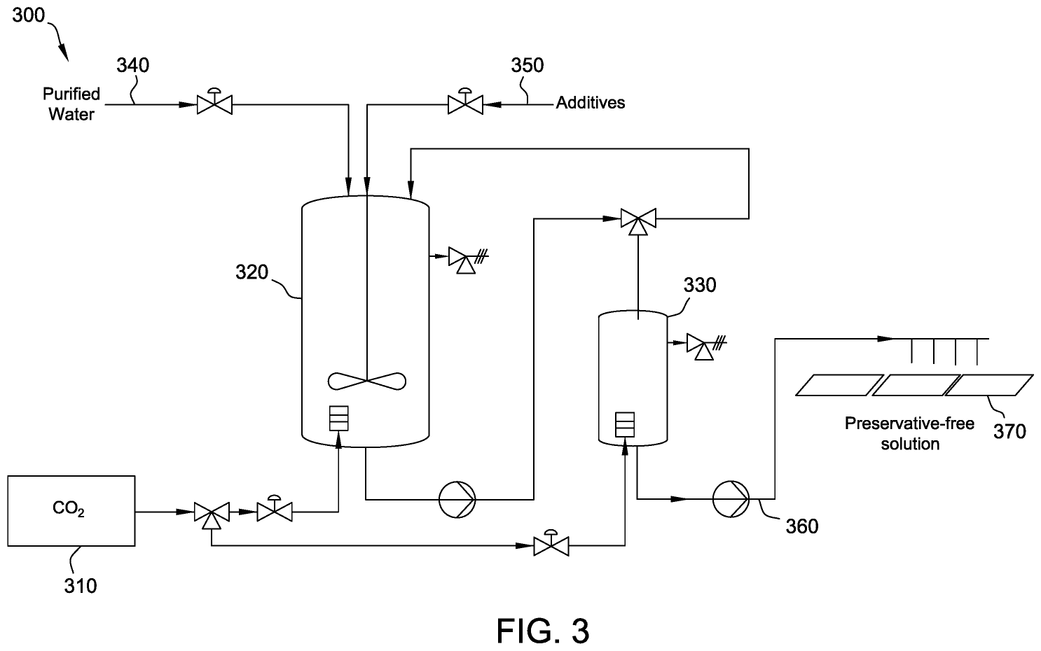

The pressurized gas comprising an inert gas may also be added directly to a mixing tank and likewise in formulating the wet wipe formulation. FIG. 3 shows an exemplary schematic of a manufacturing process 300 for a wet wipe formulation. A pressurized gas composition comprising carbon dioxide 310 is injected directly into a solution mix tank 320 and buffer tank 330 used in manufacturing the wet wipe formulation of the present disclosure. A purified water stream 340 and additives 350 as described herein may also be added to the solution mix tank 320 to yield a wet wipe formulation. In some embodiments, the wet wipe formulation is cycled between the solution mix tank 320 and buffer tank 330. In some embodiments, the final wet wipe formulation 360 is applied from the buffer tank 330 to a wet wipe substrate 370.

Advantageously, solubilizing the pressurized gas composition comprising an inert gas does not negatively impact the reverse osmosis or filtration performance, and in some embodiments allows for elimination of microbial contamination in the process water. In some embodiments, solubilizing the pressurized gas composition comprising an inert gas prior to reverse osmosis or filtration steps increases the longevity of the filters and water system due to decreased biofilm formation and fouling. This allows for easier and less frequent inline cleaning and passivation.

The pressurized gas composition comprising an inert gas is also compatible with UV treatment to remove total organic carbon.

In some embodiments, process control measurements may be installed that measure the amount of solubilized gas, allowing for modulating the levels of solubilized gas in the water, if required.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever.

### Example 1. Reduction of Bacterial Load in Reverse Osmosis Water and a Preservative-Free Wet Wipe Solution

Burkholderia cepacia (ATCC 25416) was grown overnight in 40 mL trypticase soy broth in 250 mL bottle shaken at 100 RPM at 30° C. 20 μl of culture was added to 100 mL unpreserved wet wipe solution (NB6-162C) or 100 mL reverse osmosis water (final concentration approx. 1×10⁶ or 5×10¹ CFU/mL). 100 mL of either solution were placed in a whipped cream dispenser (670 mL total volume container), and then different amounts of carbon dioxide (CO₂) were injected into the container.

100 mL of each solution were then placed in a 150 mL sterile plastic bottle (untreated control). Both samples were placed on a wrist action shaker for 2 hr at 100 rpm at room temperature (25° C.), and then placed on an orbital shaker for 70 hr at 100 rpm at room temperature (25° C.). The total time each sample was at room temperature (25° C.) was 72 hr. After 72 hr, each sample was plated on trypticase soy agar to enumerate bacterial growth after contact period. The resulting bacterial growth for each solution is shown in Tables 1-5. The conditions in the whipped cream dispenser are shown in Table 6.

TABLE 1
Reduction of bacterial load by adding 8.2 g of CO₂ to
100 mL reverse osmosis water contaminated with approximately
1 × 10⁶ CFU/mL Burkholderia cepacia (n = 3).

| Recovered Bacteria | With CO₂ | Without CO₂ |
|---|---|---|
| Average CFU/mL | 1.1 × 10³ | 1.5 × 10⁶ |
| Standard Deviation | 7.6 × 10¹ | 6.7 × 10⁴ |

TABLE 2
Reduction of bacterial load by adding 8.2 g of CO₂ to
100 mL reverse osmosis water contaminated with approximately
5 × 10¹ CFU/mL Burkholderia cepacia (n = 3).

| Recovered Bacteria | With CO₂ | Without CO₂ |
|---|---|---|
| Average CFU/mL | <10 | 5.7 × 10¹ |
| Standard Deviation | n/a | 2.1 × 10¹ |

TABLE 3
Reduction of bacterial load by adding 16.0 g of CO₂ to
100 mL reverse osmosis water contaminated with approximately
1 × 10⁵ CFU/mL Burkholderia cepacia (n = 3).

| Recovered Bacteria | With CO₂ | Without CO₂ |
|---|---|---|
| Average CFU/mL | <10 | 3.0 × 10⁵ |
| Standard Deviation | n/a | 7.6 × 10³ |

TABLE 4
Reduction of bacterial load by adding 23.7 g of CO₂ to
100 mL reverse osmosis water contaminated with approximately
1 × 10⁵ CFU/mL Burkholderia cepacia (n = 3).

| Recovered Bacteria | With CO₂ | Without CO₂ |
|---|---|---|
| Average CFU/mL | <10 | 3.4 × 10⁵ |
| Standard Deviation | n/a | 1.3 × 10⁴ |

TABLE 5
Reduction of bacterial load by adding 8.2 g of CO₂ to 100 mL
of a preservative-free wet wipe formulation contaminated with
approximately 1 × 10⁶ CFU/mL Burkholderia cepacia (n = 3).

| Recovered Bacteria | With CO₂ | Without CO₂ |
|---|---|---|
| Average CFU/mL | <10 | 3.1 × 10⁶ |
| Standard Deviation | n/a | 2.8 × 10⁵ |

TABLE 6
Conditions in the pressurized vessel.

| Added CO₂ (mol) | Vessel Pressure (atm) | CO₂ in water (mol/L) | pH |
|---|---|---|---|
| 0.17 | 7.8 | 0.265 | 3.47 |
| 0.36 | 15.4 | 0.524 | 3.32 |
| 0.54 | 20.6 | 0.702 | 3.26 |

The addition of pressurized gas comprising carbon dioxide to contaminated water greatly reduced microbial growth, compared to contaminated water where pressurized gas comprising carbon dioxide was not added.

The addition of pressurized gas comprising carbon dioxide to a contaminated preservative-free wet wipe formulation also greatly reduced microbial growth, compared to a contaminated preservative-free wet wipe formulation where pressurized gas comprising carbon dioxide was not added.

### Example 2. Reduction of Bacterial Load by Continuous Addition of CO₂

This example demonstrates the use of different concentrations of CO₂ for reducing bacterial load in reverse osmosis treated water and the broad-spectrum performance of CO₂ for reducing bacterial load in reverse osmosis treated water.

Each microbe was grown in specified growth conditions (Table 7) using 40 mL of broth media in a sterile 250 mL bottle shaken at 100 RPM. Each microbe was subcultured from broth culture to an agar plate and incubated in specified growth conditions (Table 7).

TABLE 7
Tested microorganisms and their incubation conditions.

| | | Temperature | Time |
|---|---|---|---|
| Species & Strain | Growth Medium | (° C.) | (Hours) |
| Burkholderia cepacia ATCC 25416 | Trypticase soy agar | 30 | 24 |
| Candida albicans ATCC 10231 | Sabouraud dextrose agar | 26 | 48-72 |
| Escherichia coli ATCC 8739 | Nutrient agar | 37 | 24 |
| Pseudomonas aeruginosa ATCC 9027 | Nutrient agar | 37 | 24 |
| Staphylococcus aureus ATCC 6538 | Trypticase soy agar | 37 | 24 |

100 μL of bacterial or fungal suspension matching the turbidity of a 0.5 or 1 MacFarland Standard were added to 100 mL of filter-sterilized reverse osmosis water (final concentration approximately 1×10⁶ or 5×10¹ CFU/mL).

20 mL of reverse osmosis water solution with bacteria was dispensed to a 25 mL hydrothermal synthesis autoclave reactor vessel (25 mL total volume vessel) attached to a continuous CO₂ delivery system. The vessels were left connected to the CO₂ delivery system at specific contact times at room temperature (25-27° C.).

20 mL of reverse osmosis water with bacteria and filter sterilized reverse osmosis water (no bacteria) were placed in a 150 mL sterile plastic bottle (untreated controls). The untreated controls were left for the same contact time at room temperature (25-27° C.) as CO₂ treated vessels.

Each microorganism was plated on specified agar media (Table 7) to enumerate bacteria after contact period. Tables 8-10 show the bacterial load reduction achieved after continuous addition of pressurized gas comprising CO₂ to contaminated reverse osmosis water or a contaminated solution of reverse osmosis water and Triton X-100.

TABLE 8
Reduction of bacterial load over a range of contact times by continuously adding
300 psi of CO₂ (approximately 0.694 mol/L or 20.4 atmospheres) to 20 mL reverse
osmosis water contaminated with Burkholderia cepacia (n = 2).

| Time | Average Recovered CFU/mL (Standard Deviation) | | |
|---|---|---|---|
| (Hours) | Starting Inoculum | With CO₂ | Without CO₂ |
| 0.5 | 7.0 × 10⁵ (2.0 × 10⁵) | 5.0 × 10⁵ (1.0 × 10⁵) | 6.5 × 10⁵ (5.0 × 10⁴) |
| 1 | 1.2 × 10⁶ (2.0 × 10⁵) | 4.5 × 10⁵ (2.5 × 10⁵) | 9.5 × 10⁵ (2.5 × 10⁵) |
| 1.5 | 4.0 × 10⁶ (1.0 × 10⁶) | 5.0 × 10⁵ (2.0 × 10⁵) | 3.2 × 10⁶ (4.0 × 10⁵) |
| 2 | 4.0 × 10⁶ (1.0 × 10⁶) | 3.1 × 10⁴ (7.0 × 10³) | 3.2 × 10⁶ (4.0 × 10⁵) |
| 2.5 | 4.0 × 10⁶ (1.0 × 10⁶) | 8.0 × 10³ (2.0 × 10³) | 3.2 × 10⁶ (4.0 × 10⁵) |
| 3 | 1.2 × 10⁶ (2.0 × 10⁵) | <1 (n/a) | 9.5 × 10⁵ (2.5 × 10⁵) |
| 6 | 1.2 × 10⁶ (2.0 × 10⁵) | <1 (n/a) | 9.5 × 10⁵ (2.5 × 10⁵) |
| 24 | 7.5 × 10⁵ (5.0 × 10⁴) | <1 (n/a) | 5.0 × 10⁵ (2.0 × 10⁵) |

TABLE 9
Reduction of bacterial load by adding different CO² pressures to 20
mL reverse osmosis water contaminated with Burkholderia cepacia
(n = 2). The CO2 pressure of 100 psi was approximately 0.231 mol/L or
6.8 atmospheres (atm), 200 psi was approximately 0.463 mol/L or 13.6
atm, and 300 psi was approximately 0.694 mol/L or 20.4 atm of CO₂.

| Added CO₂ | Time | Average Recovered CFU/mL (Standard Deviation) | | |
|---|---|---|---|---|
| (Psi) | (Hours) | Starting Inoculum | With CO₂ | Without CO₂ |
| 100 | 2 | 2.5 × 10⁴ (5.0 × | 2.3 × 10⁴ (1.0 × | 2.3 × 10⁴ (7.0 × |
| | | 10³) | 10³) | 10³) |
| | 4 | 2.5 × 10⁴ (5.0 × | 1.2 × 10⁴ (4.0 × | 2.3 × 10⁴ (7.0 × |
| | | 10³) | 10³) | 10³) |
| | 6 | 2.5 × 10⁴ (5.0 × | 1.2 × 10⁴ (3.5 × | 2.3 × 10⁴ (7.0 × |
| | | 10³) | 10³) | 10³) |
| 200 | 2 | 1.4 × 10⁴ (1.0 × | 1.2 × 10³ (1.5 × | 6.0 × 10³ (2.0 × |
| | | 10³) | 10²) | 10³) |
| | 4 | 1.4 × 10⁴ (1.0 × | 1.0 × 10² (1.0 × | 6.0 × 10³ (2.0 × |
| | | 10³) | 10²) | 10³) |
| | 6 | 1.4 × 10⁴ (1.0 × | 1.4 × 10¹ (<10) | 6.0 × 10³ (2.0 × |
| | | 10³) | | 10³) |
| | 2 | 5.5 × 10³ (5.0 × | 9.0 × 10² (1.0 × | 6.0 × 10³ (2.0 × |
| | | 10²) | 10²) | 10³) |
| 300 | 4 | 5.5 × 10³ (5.0 × | 5.0 × 10¹ (5.0 × | 6.0 × 10³ (2.0 × |
| | | 10²) | 10¹) | 10³) |
| | 6 | 5.5 × 10³ (5.0 × | <10 (n/a) | 6.0 × 10³ (2.0 × |
| | | 10²) | | 10³) |

Table 10. Reduction of microorganism load by continuously adding 300 psi of CO₂ (approximately 0.694 mol/L or 20.4 atmospheres) to 20 mL reverse osmosis water contaminated with various microorganisms (n=2).

TABLE 10
Reduction of microorganism load by continuously adding 300 psi of
CO₂ (approximately 0.694 mol/L or 20.4 atmospheres) to 20 mL reverse
osmosis water contaminated with various microorganisms (n = 2).

| | Average Recovered CFU/mL | | | | |
|---|---|---|---|---|---|
| | | With | | | Without |
| | Starting | CO₂ | | | CO₂ |
| Organism | Inoculum | 2 Hours | 4 Hours | 6 Hours | 6 Hours |
| Burkholderia cepacia ATCC | 1.0 × 10⁴ | 1.3 × 10³ | 2.5 × 10¹ | <10 | 3.5 × 10³ |
| 25416 | | | | | |
| Candida albicans ATCC | 2.5 × 10⁴ | 5.3 × 10² | 1.0 × 10² | <10 | 4.5 × 10³ |
| 10231 | | | | | |
| Escherichia coli ATCC | 1.7 × 10⁴ | 1.0 × 10³ | 2.5 × 10² | <10 | 6.0 × 10³ |
| 8739 | | | | | |
| Pseudomonas aeruginosa | 2.5 × 10⁴ | 3.5 × 10² | <10 | <10 | 3.5 × 10⁴ |
| ATCC 9027 | | | | | |
| Staphylococcus aureus ATCC | 7.0 × 10⁴ | 6.0 × 10² | 1.3 × 10² | <10 | 4.5 × 10⁴ |
| 6538 | | | | | |

TABLE 11
Reduction of bacterial load by continuously adding 100 psi of CO₂
(approximately 0.231 mol/L or 6.8 atmospheres) to 20 mL reverse
osmosis water and 0.2% (v/v) Triton X-100 solution contaminated
with approximately 1.0 × 10⁴ Burkholderia cepacia (n = 2).

| | |
|---|---|
| Time | Average Recovered CFU/mL |

| (Minutes) | With CO₂ | Without CO₂ |
|---|---|---|
| 15 | 2.1 × 10³ | n/a |
| 30 | 1.2 × 10³ | n/a |
| 60 | 6.3 × 10² | 2.5 × 10³ |

This written description uses examples to illustrate the present disclosure, including the best mode, and also to enable any person skilled in the art to practice the disclosure, including making and using any compositions or systems and performing any incorporated methods. The patentable scope of the disclosure is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

What is claimed is:

1. An antimicrobial wet wipe composition comprising:
a wet wipe formulation comprising:
an aqueous solution;
a pressurized gas composition comprising an inert gas solubilized in the aqueous solution; and
optionally, at least one additive.

2. The wet wipe composition of claim 1, wherein the antimicrobial wet wipe composition comprises a wet wipe substrate.

3. The wet wipe composition of claim 1, wherein the inert gas is selected from the group consisting of carbon dioxide, nitrogen, argon, helium, and mixtures thereof.

4. The wet wipe composition of claim 1, wherein the pressurized gas is at a pressure in a range of from about 15 psi to about 300 psi.

5. The wet wipe composition of claim 1, wherein the pressurized gas is at a pressure in a range of from about 15 psi to about 100 psi.

6. The wet wipe composition of claim 1, wherein a concentration of the inert gas in the aqueous solution is in a range of from about 0.01 mol/L to about 0.7 mol/L.

7. The wet wipe composition of claim 1, wherein a concentration of the inert gas in the aqueous solution is in a range of from about 0.01 mol/L to about 0.3 mol/L.

8. The wet wipe composition of claim 1, wherein the wet wipe formulation does not comprise a traditional or non-traditional preservative.

9. A consumer product comprising the antimicrobial wet wipe composition of claim 1.

10. A method of preserving a wet wipe formulation, the method comprising:
solubilizing a pressurized gas composition comprising an inert gas in the wet wipe formulation.

11. The method of claim 10, wherein the inert gas is selected from the group consisting of carbon dioxide, nitrogen, argon, helium, and mixtures thereof.

12. The method of claim 10, wherein the wet wipe formulation does not comprise a traditional or non-traditional preservative.

13. The method of claim 10, wherein the pressurized gas is at a pressure in a range of from about 15 psi to about 300 psi.

14. The method of claim 10, wherein a concentration of the inert gas in the wet wipe formulation is in a range of from about 0.01 mol/L to about 0.7 mol/L.

15. A method of manufacturing a wet wipe formulation, the method comprising:
solubilizing a pressurized gas composition comprising an inert gas in an aqueous solution; and
adding the aqueous solution to the wet wipe formulation.

16. The method of claim 15, wherein the inert gas is selected from the group consisting of carbon dioxide, nitrogen, argon, helium, and mixtures thereof.

17. The method of claim 15, wherein the aqueous solution comprises the pressurized gas at a pressure in a range of from about 15 psi to about 300 psi.

18. The method of claim 15, wherein a concentration of the inert gas in the aqueous solution is in a range of from about 0.01 mol/L to about 0.7 mol/L.

19. The method of claim 15, wherein the method further comprises a step of applying filtration, reverse osmosis, or UV treatment to the aqueous solution.

20. The method of claim 15, wherein the method does not comprise a step of adding a chemical preservative to the aqueous solution.
